(19) United States
(12) Patent Application Publication
(10) Pub. No.: US 2023/0051982 A1
(43) Pub. Date: Feb. 16, 2023

(54) METHODS AND SYSTEMS FOR LONGITUDINAL PATIENT INFORMATION PRESENTATION

(71) Applicant: GE Precision Healthcare LLC, Wauwatosa, WI (US)

(72) Inventors: Sanand Sasidharan, Bengaluru (IN); Ravi Bhardwaj, Bengaluru (IN); Anuradha Kanamarlapudi, Bengaluru (IN); Raghu Prasad, Bengaluru (IN)

(21) Appl. No.: 17/819,496

(22) Filed: Aug. 12, 2022

(30) Foreign Application Priority Data
Aug. 13, 2021 (IN) 202141036677

(51) Int. Cl. G16H 15/00; G16H 10/60

(52) U.S. Cl. CPC G16H 15/00; G16H 10/60

(57) ABSTRACT

Various methods and systems are provided for longitudinal presentation of patient information. In one example, a computing device comprises a display screen, the computing device being configured to display on the screen a timeline of patient medical information including a plurality of symbols representing the patient medical information, wherein a symbol of the plurality of symbols is selectable to launch a details panel and enable a report that references the displayed patient medical information to be seen within the timeline, and wherein the symbol is displayed while the details panel is in an un-launched state.

### RELATED APPLICATIONS

This application claims priority to Indian Provisional Application No. 202141036677, entitled METHODS AND SYSTEMS FOR LONGITUDINAL PATIENT INFORMATION PRESENTATION, and filed Aug. 13, 2021, the entire contents of which is hereby incorporated by reference for all purposes.

### FIELD

Embodiments of the subject matter disclosed herein relate to presentation of patient information, and more particularly to a platform for presenting a visual, longitudinal timeline of patient information.

### BACKGROUND

Digital collection, processing, storage, and retrieval of patient medical records may include a conglomeration of large quantities of data. In some examples, the data may include numerous medical procedures and records generated during investigations of the patient, including a variety of examinations, such as blood tests, urine tests, pathology reports, image-based scans, and so on. Duration of the diagnosis of a medical condition of a subject followed by treatment may be spread over time from few days to few months or even years in the case of chronic diseases, which may be diseases that take more than one year to cure. Over the course of diagnosing and treating chronic disease, the patient may undergo many different treatments and procedures and may move to different hospitals and/or geographic locations.

Physicians are increasingly relying on Electronic Medical Record (EMR) systems to record and review historical health records of the patient during diagnosis, treatment, and monitoring of a patient condition. For patients with chronic illnesses, there are often hundreds or even thousands of EMRs resulting from numerous visits. Sorting and extracting information from past EMRs for such patients is a slow and inefficient process, increasing a likelihood of missing records with relevant data which may be spread out across a large number of less informative routine visit records.

### BRIEF DESCRIPTION

In one embodiment, a computing device comprises a display screen, the computing device being configured to display on the screen a timeline of patient medical information including a plurality of symbols representing the patient medical information, wherein a symbol of the plurality of symbols is selectable to launch a details panel and enable a report that references the displayed patient medical information to be seen within the timeline, and wherein the symbol is displayed while the details panel is in an un-launched state.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, wherein below:

FIG. 1 illustrates a system for displaying clinical information of a patient to a user in accordance with an aspect of the disclosure.

FIG. 2 shows a first example patient timeline generated with the system of FIG. 1.

FIG. 3 shows a second example patient timeline generated with the system of FIG. 1.

FIG. 4 shows a segment of a timeline obtained via a natural language temporal search query.

FIG. 5 shows an example transformation of siloed data and decisions into an integrated care pathway.

FIG. 6 shows a third example patient timeline generated with the system of FIG. 1.

FIG. 7 schematically shows a process for leveraging the system of FIG. 1 to improve data access and integration for a multi-disciplinary team.

FIG. 8 shows a process for utilizing the system of FIG. 1 to generate longitudinal data elements for a patient for display to a user.

FIG. 9 shows a method for navigating to a segment of a patient information timeline based on a natural language input.

FIG. 10 shows a method for modifying a patient information timeline based on a user specialization.

FIG. 11 shows a method for calculating an at-home infusion risk score for a patient.

FIG. 12 shows a method for facilitating enhanced report and/or record generation.

FIG. 13 shows an example process for generating a report using a report generation model.

FIG. 14 shows an example unfilled table of domain ordering constraints.

FIG. 15A, FIG. 15B, and FIG. 15C each show example filled tables of domain ordering constraints.

### DETAILED DESCRIPTION

The following description relates to various embodiments of patient history analysis and display of longitudinal patient information that structures a patient's medical data into a visual longitudinal patient journey view that aids clinical thinking and guides actions to achieve efficiency and personalized patient experience.

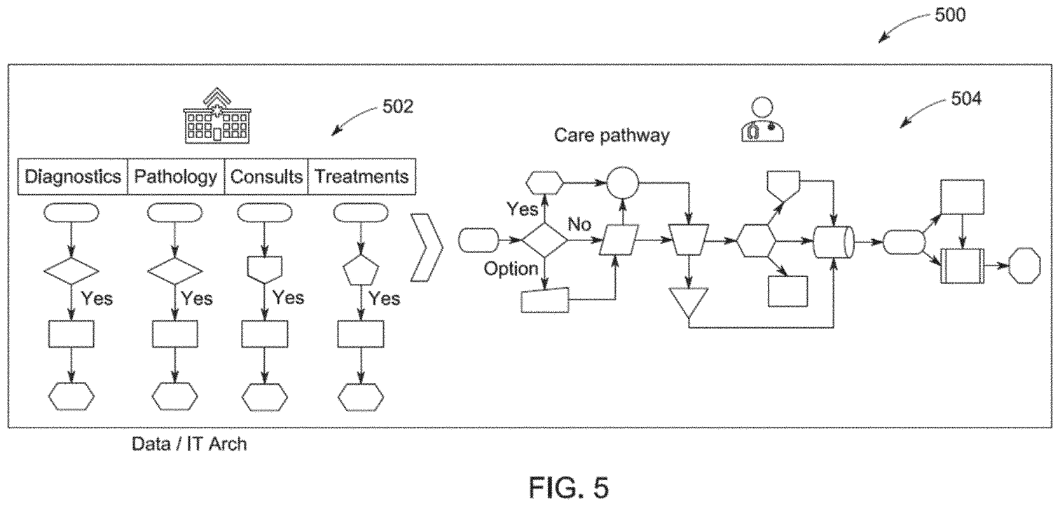

For example, FIG. 5 shows a schematic representation 500 of a transformation of siloed data 502 into an integrated care pathway 504. As will be explained in more detail below, different types of medical data may be stored in different locations, accessed via different interfaces, and used in different ways to make decisions about patient care. As shown in FIG. 5, the siloed data 502 may include diagnostics, pathology, consults, and treatments. Previous approaches of viewing and considering such data independently when making patient care decisions may lead to overlooked information that may affect a treatment decision, for example, and may increase time burden and mental load placed on the clinicians caring for a patient. According to embodiments disclosed herein, the siloed data may be integrated into the care pathway, where relevant data for a patient condition (e.g., cancer) may be viewed at one time, on a single view. The different aspects of a patient's medical data may be considered together when developing treatment plans, thus improving patient outcomes and the efficiency of the clinicians.

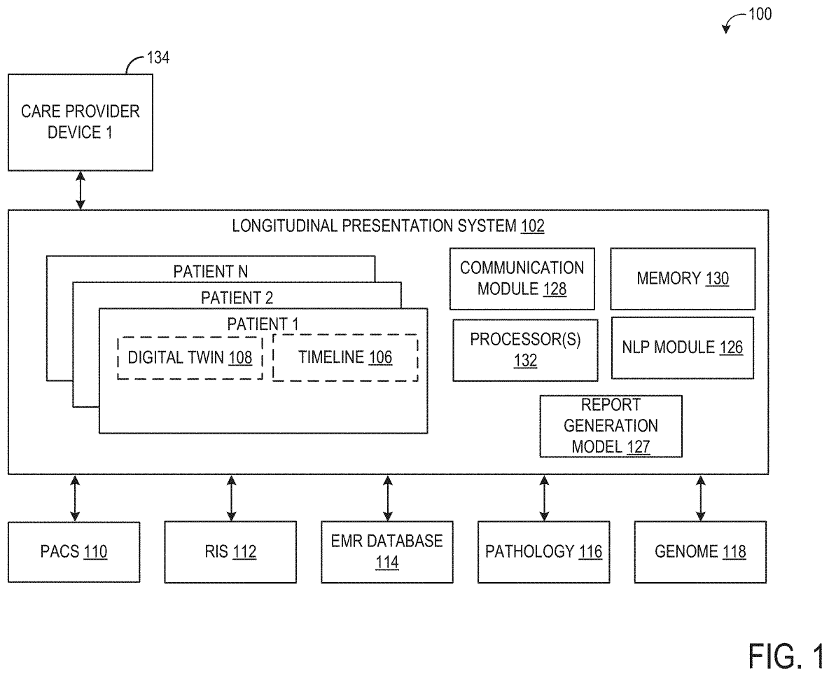

Embodiments of the present disclosure will now be described, by way of example, with reference to the figures, in which FIG. 1 schematically shows an example patient information system 100 that may be implemented in a medical facility such as a hospital. Patient information system 100 may include a longitudinal presentation system 102. Presentation system 102 may include resources (e.g., memory 130, processor(s) 132) that may be allocated to store and execute timelines and a digital twin for each of a plurality of patients. For example, as shown in FIG. 1, timeline 106 and digital twin 108 are stored on presentation system 102 for a first patient (patient 1); a plurality of additional timelines and digital twins may be stored on and/or generated by presentation system 102, each corresponding to a respective patient (patient 2 up to patient N).

Each timeline 106 may include graphical representations of patient medical events arranged chronologically. The patient medical events depicted on the timeline 106 may include office or hospital visits (and information gathered during such visits), findings from diagnostic imaging, pathology reports, lab test results, biomarker testing results, and any other clinically relevant information. Further, the patient medical information, including medical history, current state, vital signs, and other information, may be entered to the digital twin 108, which may be used to gain situational awareness, clinical context, and medical history of the patient to facilitate predicted patient states, procurement of relevant treatment guidelines, patient state diagnoses, etc., which may be used to generate the timelines disclosed herein and/or included as part of the timelines disclosed herein.

The patient information that is presented via the timeline 106 may be stored in different medical databases or storage systems in communication with presentation system 102. For example, as shown, the presentation system 102 may be in communication with a picture archiving and communication system (PACS) 110, a radiology information system (RIS) 112, an EMR database 114, a pathology database 116, and a genome database 118. PACS 110 may store medical images and associated reports (e.g., clinician findings), such as ultrasound images, MRI images, and so on. PACS 110 may store images and communicate according to the DICOM format. RIS 112 may store radiology images and associated reports, such as CT images, X-ray images, and so on. EMR database 114 may store electronic medical records for a plurality of patients. EMR database 114 may be a database stored in a mass storage device configured to communicate with secure channels (e.g., HTTPS and TLS), and store data in encrypted form. Further, the EMR database is configured to control access to patient electronic medical records such that only authorized healthcare providers may edit and access the electronic medical records. An EMR for a patient may include patient demographic information, family medical history, past medical history, lifestyle information, preexisting medical conditions, current medications, allergies, surgical history, past medical screenings and procedures, past hospitalizations and visits, and so on. Pathology database 116 may store pathology images and related reports, which may include visible light or fluorescence images of tissue, such as immunohistochemistry (IHC) images. Genome database 118 may store patient genotypes (e.g., of tumors) and/or other tested biomarkers.

Presentation system 102 may aggregate data received from PACS 110, RIS 112, EMR database 114, pathology database 116, genome database 118, and/or any other connected patient data sources and generate timelines from the aggregated data. For example, for patient 1, the aggregated data associated with that patient may be saved in the digital twin 108. In some examples, the data may be processed before the data is saved in the digital twin, such that only filtered or otherwise relevant patient data is saved in the digital twin. In some examples, when timeline 106 is generated, the presentation system 102 may query the various data sources (e.g., PACS 110, RIS 112, EMR database 114, pathology database 116, genome database 118, and/or any other connected patient data sources) to retrieve data for patient 1. The data may be saved in the digital twin 108 so that the data is available for future iterations of the timeline for patient 1. However, in other examples, the data sources may occasionally push the data to the presentation system and/or the data may not be permanently saved in the presentation system 102 (e.g., the data may be cached for the purposes of generating the timeline but then removed once the timeline has been generated or after a predetermined amount of time has passed since the timeline was generated).

When requested, timeline 106 may be displayed on one or more display devices. As shown in FIG. 1, a care provider device 134, and in some examples more than one care provider device, may be communicatively coupled to presentation system 102. Each care provider device may include a processor, memory, communication module, user input device, display (e.g., screen or monitor), and/or other subsystems and may be in the form of a desktop computing device, a laptop computing device, a tablet, a smart phone, or other device. Each care provider device may be adapted to send and receive encrypted data and display medical information, including medical images in a suitable format such as digital imaging and communications in medicine (DICOM) or other standards. The care provider devices may be located locally at the medical facility (such as in the room of a patient or a clinician's office) and/or remotely from the medical facility (such as a care provider's mobile device).

When viewing timeline 106 via a display of a care provider device, a care provider may enter input (e.g., via the user input device, which may include a keyboard, mouse, microphone, touch screen, stylus, or other device) that may be processed by the care provider device and sent to the presentation system 102. In examples where the user input is a selection of a link or user interface control button of the timeline, the user input may trigger display of a selected EMR, trigger progression to a desired point in time or view of the timeline (e.g., trigger display of desired patient medical information), trigger updates to the configuration of the timeline, or other actions.

In some examples, presentation system 102 may include a natural language processing (NLP) module 126. NLP module 126 may analyze human voice and text communication to obtain/infer various information related to the patient history, clinical queries, and so on. In doing so, NLP module 126 serves as a monitor, by listening to the events in the clinician and patient surroundings including medical staff conversations and patient input. The monitored conversations/inputs may be used to record the patient's status (for EMR/digital twin) or to infer clinician reasoning. The NLP module 126 may receive output from one or more microphones positioned in proximity to the patient, for example, in order to monitor the conversations and inputs. The NLP module 126 may also analyze text-based inputs and data, such as clinician queries entered via text-based user input and the aggregated patient data included in the digital twin (e.g., received from the patient data sources, such as the PACS 110 and the EMR database 114).

The presentation system 102 may be configured to receive queries from care providers and utilize natural language processing to determine what information is being requested in the queries. For example, the NLP module 126 may utilize natural language processing to determine if a query includes a request to view a timeline, a specific portion of the timeline, or more detailed information of an event in the timeline, and if so, determine what information is being requested. The NLP module 126 may execute deep learning models (e.g., machine learning or other deep learning models such as neural networking) or other models that are trained to understand medical terminology. Further, the deep learning models may be configured to learn updates or modifications to the models in an ongoing manner in a patient and/or care provider specific manner.

In a first example, the NLP module 126 may follow a rule-based approach such that it is configured with a set of answers for predetermined, likely questions. When a question is received, the NLP module 126 may be configured to output an answer from the set of answers. In a second example, the NLP module 126 may use a directed acyclic graphs (DAG) of states, each of which include rules for how to react and how to proceed to various questions. Thus, the NLP module 126 described herein may include artificial intelligence and be adapted to handle natural language which is a way to take human input and map it to intent and entities. The NLP module 126 may be adapted to hold a state and map the state with (intent, entities) to an actionable application programming interface (API). The mapping may be performed by teaching machine learning models by providing the models with examples of such mappings.

In some examples, the NLP module 126 may receive patient input from a microphone (e.g., patient speech) and identify the cancer-related (or other condition) patient-reported-outcome being mentioned by the patient via speech and when the patient interacts with a clinician. The outcome may be segregated into disease-related, treatment-related, and non-related categories, entered into the patient's EMR and/or digital twin, and included on the timeline.

The NLP module 126 may further be used to generate the timelines disclosed herein (e.g., timeline 106). For example, the NLP module 126 may analyze text from a patient report/EMR in order to extract and/or summarize relevant information from the text to be included in the timeline. To accomplish this, the NLP module 126 may perform entity recognition on the text. Entity recognition may include identifying entities from the text, such as a type of tumor, a position of the tumor, and a body part at which the tumor is located. The NLP module 126 may also perform assertion recognition where the NLP module 126 may identify positive and negative assertions of clinical markers, such as presence or absence of symptoms, from the text. Further, the NLP module 126 may perform relation recognition, where relationships between keywords in the text may be identified. For example, relation recognition may include recognizing a relationship between the identified tumor and the body part as “in to”, and a relationship between the identified tumor and the tumor position as “at.”

The NLP module 126 may also perform ontology linking where concepts and categories within a domain, such as a health condition or a disease, may be recognized and paired from the text. As such, the NLP module 126 may be configured to recognize and generate binary relationships between clinical terminology and codes. As one example, the text of the EMR may be scanned for coded terms according to a type of medical coding and the NLP module 126 may correlate a medical diagnosis code to the coded terms. An example of a coded term may be a “nodular tumor extension,” which may be linked to a medical diagnosis code of “385413003” from SNOMED Clinical Terms (e.g., a computer-processable collection of medical terms including codes, terms, synonyms, and definitions). As another example, the coded term may be the tumor position, such as “8-10 o'clock,” which may be correlated to a RadLex code of “RID6028,” where RadLex is set of radiology terms. As yet another example, the coded term may be the location of the tumor, e.g., “mesorectal fat,” which may correspond to a NCIT code of “C25565,” where NCIT is a standard for biomedical coding and reference. By identifying the medical diagnosis codes linked to the coded term, the NLP module 126 may parse medical information associated with the medical diagnosis codes from documents and/or databases accessible by the presentation system.

Finally, clinical markers may be recognized, e.g., clinical marker recognition, and extracted from the text (or the text as processed by the NLP module 126, such as after the entity recognition, assertion recognition, relation recognition, and/or ontology linking are performed. For example, all clinical markers may be identified and extracted from the EMR by the NLP module 126 and the clinical markers may be listed in the timeline and/or relevant text from the EMR surrounding the clinical markers may be included in the timeline.

In some examples, the presentation system 102 may include a report generation model 127 that may be configured to generate patient-customized report templates and/or make suggestions to a clinician for what patient parameters should be tracked and entered for each patient report. The report generation model 127 may include one or more machine learning models, such as neural networks, that are trained to identify a current path of the patient condition and provide parameters to be included in the patient's report based on the current path of the patient. The report generation model 127 may be trained and validated off-line and the validated, trained model may be stored in memory of the presentation system 102.

In some embodiments, a management application executed by the presentation system 102 may allow an administrator to configure how the timelines are displayed, what information is conveyed by the timelines for each patient, and so on. The management application may include an interface for configuring hospital specific protocols and guidelines for generating and displaying the timelines.

Presentation system 102 includes a communication module 128, memory 130, and processor(s) 132 to store and generate the timelines and digital twins, as well as send and receive communications, graphical user interfaces, medical data, and other information. Communication module 128 facilitates transmission of electronic data within and/or among one or more systems. Communication via communication module 128 can be implemented using one or more protocols. In some examples, communication via communication module 128 occurs according to one or more standards (e.g., Digital Imaging and Communications in Medicine (DICOM), Health Level Seven (HL7), ANSI X12N, etc.). Communication module 128 can be a wired interface (e.g., a data bus, a Universal Serial Bus (USB) connection, etc.) and/or a wireless interface (e.g., radio frequency, infrared, near field communication (NFC), etc.). For example, communication module 128 may communicate via wired local area network (LAN), wireless LAN, wide area network (WAN), and so on using any past, present, or future communication protocol (e.g., BLUETOOTH™, USB 2.0, USB 3.0, etc.).

Memory 130 may include one or more data storage structures, such as optical memory devices, magnetic memory devices, or solid-state memory devices, for storing programs and routines executed by processor(s) 132 to carry out various functionalities disclosed herein. Memory 130 may include any desired type of volatile and/or non-volatile memory such as, for example, static random access memory (SRAM), dynamic random access memory (DRAM), flash memory, read-only memory (ROM), and so on.

Processor(s) 132 may be any suitable processor, processing unit, or microprocessor, for example. Processor(s) 132 may be a multi-processor system, and, thus, may include one or more additional processors that are identical or similar to each other and that are communicatively coupled via an interconnection bus.

As used herein, the terms “sensor,” “system,” “unit,” or “module” may include a hardware and/or software system that operates to perform one or more functions. For example, a sensor, module, unit, or system may include a computer processor, controller, or other logic-based device that performs operations based on instructions stored on a tangible and non-transitory computer readable storage medium, such as a computer memory. Alternatively, a sensor, module, unit, or system may include a hard-wired device that performs operations based on hard-wired logic of the device. Various modules or units shown in the attached figures may represent the hardware that operates based on software or hardwired instructions, the software that directs hardware to perform the operations, or a combination thereof.

“Systems,” “units,” “sensors,” or “modules” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be off-the-shelf devices that are appropriately programmed or instructed to perform operations described herein from the instructions described above. Additionally or alternatively, one or more of these devices may be hard-wired with logic circuits to perform these operations.

Thus, presentation system 102 may be configured to obtain/ingest medical data from a variety of sources (e.g., PACS, EMR, RIS, etc.) and analyze, extract, and register selected medical data to generate a timeline for each patient as described herein. In some examples, presentation system 102 may include one or more data filters (e.g., AI-assisted data filters) configured to monitor and filter the ingested data to ensure that only relevant and complete data is presented in the timeline. In some examples, an indication of the level of confidence in the data (e.g., confidence in the relevancy and/or accuracy of the data) may be presented with an icon in each timeline. This adds to the confidence factors in a clinical solution and also leans towards being representative of precision health. This would apply to quality control checks on genomic data, image quality evaluation of digital pathology, radiology (ensuring appropriateness of protocols for the condition adjudged), and similarly carry scores from NLP ingestion of the confidence scores in data translation, characterization, and correlation.

To ensure completeness and accuracy of the presentation of the longitudinal data in each timeline, data alignment (alignment for imaging data, both radiology and pathology) may be performed to ensure pathology tracking and quantification. Each time new data is ingested, the presentation system 102 may identify the most relevant past data, localize matching structure, and visually seek verification and lock-in the co-registered data for quantitative assessment. [Each step would offer a meaningful confidence metric and aggregate the metric as the journey proceeds. The data alignment may be performed to ensure that clinical markers in text from an EMR are properly matched to one or more corresponding images (whether diagnostic images obtained by ultrasound, CT, MRI, etc., or pathology images) that illustrate the clinical markers. For example, a timeline entry may be created from an EMR that references a particular anatomical structure (e.g., a tumor) shown in diagnostic images taken at an imaging exam a day prior. The imaging exam may include a plurality of images, only some of which include the particular anatomical structure. The matching/alignment may be performed so that the timeline entry includes only those images that illustrate the particular anatomical structure. In doing so, the correct image(s) is shown.

One or more of the devices described herein may be implemented over a cloud or other computer network. For example, presentation system 102 is shown in FIG. 1 as constituting a single entity, but it is to be understood that presentation system 102 may be distributed across multiple devices, such as across multiple servers. Further, while the elements of FIG. 1 are shown as being housed at a single medical facility, it is to be appreciated that any of the components described herein (e.g., EMR database, RIS, PACS, etc.) may be located off-site or remote from the presentation system 102. Further, the longitudinal data utilized by the presentation system 102 for the timeline generation and other tasks described below could come from systems within the medical facility or obtained through electronic means (e.g., over a network) from other referring institutions.

While not specifically shown in FIG. 1, additional devices described herein (e.g., care provider device 134) may likewise include user input devices, memory, processors, and communication modules/interfaces similar to communication module 128, memory 130, and processor(s) 132 described above, and thus the description of communication module 128, memory 130, and processor(s) 132 likewise applies to the other devices described herein. As an example, the care provider devices (e.g., care provider device 134) may store user interface templates in memory that include placeholders for relevant information stored on presentation system 102 or sent via presentation system 102. For example, care provider device 134 may store a user interface template for a patient timeline that a user of care provider device 134 may configure with placeholders for desired patient information. When the timeline is displayed on the care provider device, the relevant patient information may be retrieved from presentation system 102 and inserted in the placeholders. The user input devices may include keyboards, mice, touch screens, microphones, or other suitable devices.

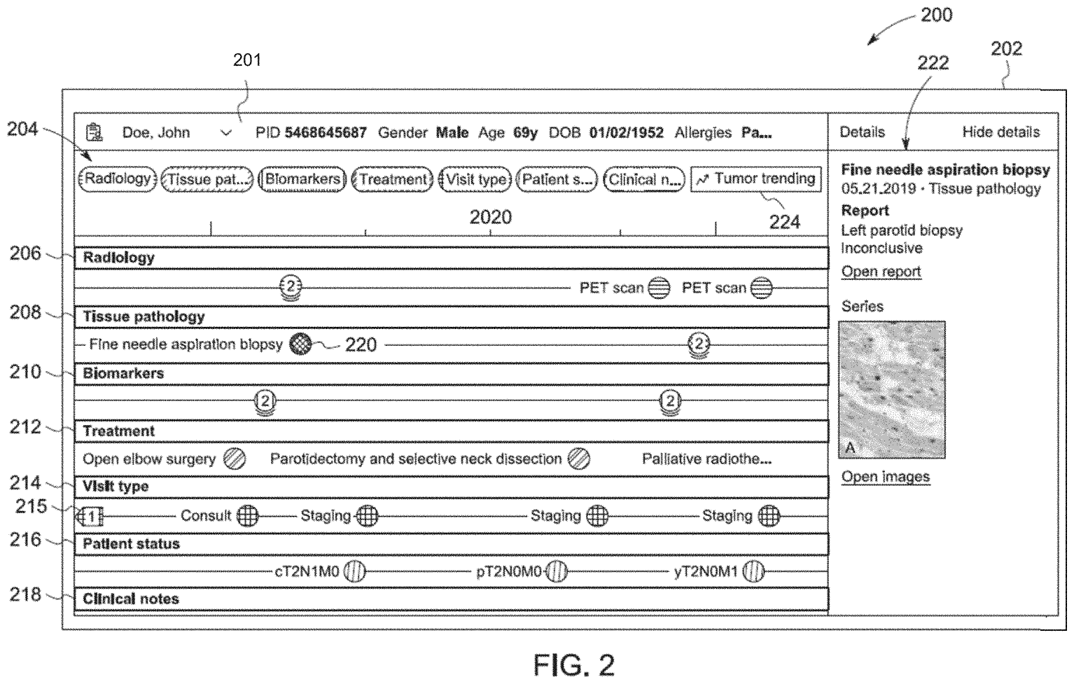

FIG. 2 shows a first example of a timeline 200 that may be generated for a patient by presentation system 102. Timeline 200 may be displayed on a display 202, which may be part of a care provider device (e.g., care provider device 134). Timeline 200 may include a plurality of different categories of timelines that may be displayed together or individually in a time-aligned manner as different swimlanes (e.g., rows) of the timeline 200. Timeline 200 includes a Quick Access Banner 201 which displays patient demographics, cancer type and stage, allergies, and ECOG status. A selection banner 204 indicates which timelines are displayed and/or allows a user to toggle each timeline category on and off so that all timelines are displayed or only or one or a subset of the timelines are displayed.

As shown, the timelines include a radiology timeline 206, a tissue pathology (e.g., tissue biopsy result) timeline 208, a protein/genomic biomarkers timeline 210, a treatment timeline 212, a visits/encounters timeline 214, a patient status/events timeline 216 (with a scroll button 215 via which more events may be displayed), and a clinical notes section 218. Each timeline includes text and/or graphical symbols to indicate events and/or patient information determined across a time frame indicated by the time bar 219 in FIG. 2. Any events or medical records in the displayed time frame (e.g., months or years) that correspond to a timeline category may be visually indicated in the corresponding swimlane/timeline, herein as a color-coded dot including a brief description of the event or record where space allows. The time-ordered series of events, records, reports, etc., may be referred to as a tuple and a tuple may be generated for each timeline category. Each tuple may include symbols/markers having the same visual appearance as other markers in that tuple with different tuples having different marker/symbol visual appearances. For example, a tissue pathology record obtained via a fine needle aspiration biopsy is indicated by dot 220, which is yellow (and other dots in the pathology timeline are also yellow). When more than one event or record is present at a given timepoint for the same timeline category/swimlane, a numeral may be displayed (e.g., 2) indicating that more than one event or record is present. Selection of dot 220 causes a details panel 222 to be displayed over a region of the timeline 200, where a representative image is shown along with findings from the biopsy and other details. At least a portion of the information included in the details panel 222 may be retrieved from/stored in the presentation system (e.g., in the digital twin), while other information included in the details panel 222 may be viewed from the original data source. For example, the details panel 222 may include links to the full pathology report and the images from the pathology report, which may be viewed from the pathology database, for example. Before dot 220 is selected, the details panel 222 may be in an un-launched state (e.g., not displayed) and the interface of the specific data source (e.g., an interface of the pathology database) may be in an un-launched state until the user selects the link in the details panel, for example. If any of the dots are selected, a similar details panel may be displayed to show relevant information and links to the full report or EMR. Timeline 200 further includes a trending button 224 that, when selected, triggers display of trends of relevant parameters (e.g., tumor trends).

While not shown in FIG. 2, in some examples, the timeline 200 may include a response to treatment visualization, where a swim-lane is included showing a small silhouette of a body/anatomical region with tumors indicated on the small silhouette. For example, tumors may be shown as circles or other shapes, and a size and/or color of the shape can be used to indicate various parameters like primary/secondary, lymph-node involvement, and so on. Treatment visualization on the timeline 200 may also show treatment parameters such as systemic/antineoplastic, irradiated site, and so on.

Thus, via timeline 200, patient information relevant to a patient condition (e.g., cancer) may be displayed in a time-ordered fashion. The patient information may be displayed via small graphical elements with minimal text, which may allow a large number or events, records, and reports to be included on the same timeline. A user may then select a graphical element of interest to view more information about the corresponding event, record, or report. The patient information may be stored in different databases that would otherwise be accessed via individual interfaces, and thus by aggregating the patient information via the timeline 200, the amount of time necessary to review relevant patient information for diagnosis and treatment decisions may be reduced.

The timelines disclosed herein aggregate patient data to a single place (e.g., into a single application) which may decrease a time used to search for known but scattered data, and unknown and missing data. Generation of the timelines may reduce cognitive overloads and aid clinical thinking for a clinician because the patient record data is reconstructed into a clinically helpful structure (co-morbidities complicates decision making). Transfer patients or new patients may be quickly diagnosed or complete treatment, as the simple multi-omic view given by the patient information timeline may assist oncologists who are on call in quickly identifying relevant patient information.

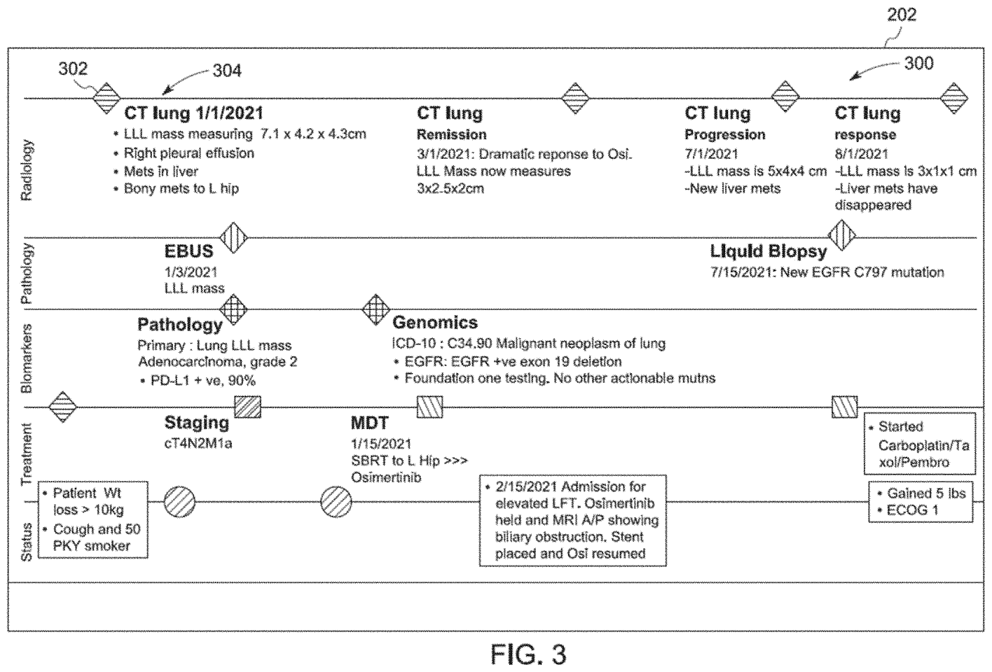

FIG. 3 shows a second example timeline 300 that may be generated for a patient by presentation system 102. Timeline 300 may be displayed on a display 202, which may be part of a care provider device (e.g., care provider device 134). Timeline 300 may be similar to timeline 200, and as such includes swimlanes for radiology, pathology, biomarkers, treatment, and status. Further, the graphical elements, such as diamond 302, included to represent the different events, reports, records, etc., may be color coded and shape-coded. Associated with each graphical element is a summary, such as summary 304, of that event, record, report, and so on. The summary may be generated automatically using NLP (e.g., with NLP module 126) to extract clinical markers from each event, report, record, etc. Selection of a graphical element may cause display of the associated record, report, etc.

While timelines 200 and 300 show events and other relevant medical information over a period of time, it may be challenging to view patient information for a given patient over a relatively long time period, due to limitations on the size of the display device. Thus, a segment of a patient timeline may typically be viewed, and the user may navigate to a desired time segment by scrolling or another user input. However, for patients with a long history, navigating to find desired information may be time-consuming. Further, the minimal nature of the timeline may make it difficult for the user to quickly identify which events, records, or reports are the most relevant or of interest for the current task. Further still, medical data has implied ordering (e.g., “resection biopsy” implies that the biopsy is after surgery) and constraints (e.g., metastasis happens after primary tumor) and thus standard key word searches may pose challenges for identifying temporal events in medical data.

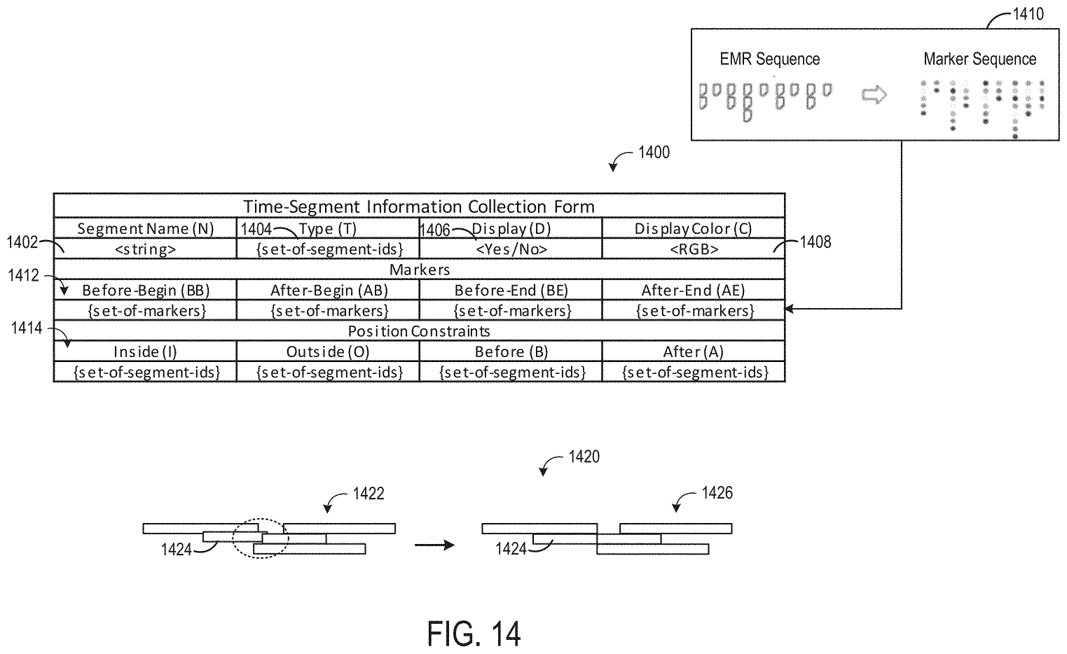

Accordingly, the NLP module 126 of the presentation system 102 may be leveraged to help the user navigate to the appropriate time-point in the patient's timeline using temporal event-related phrases. The user may input a natural language query, such as “find metastasis phase” and via the NLP module 126, the presentation system 102 may recognize a condition-specific event (or record or report) which the user has specified as input, and the event's temporal relation with other events in patient's timeline. For example, if the user wants to navigate to the metastasis phase, the presentation system 102 will make the inference that the time-period after the detection of a secondary tumor is the metastasis phase and navigate the user to that region of the timeline, which may be advantageous because each report following metastasis may not necessarily explicitly mention metastasis, and thus the temporal event-related phrase-based NLP searching described herein may identify records/reports that may be overlooked using standard keyword-based searching. One example approach to facilitate the natural temporal searching is to incorporate domain ordering and constraints via an ontology, then use this to generate training data for a machine learning. Additional details about domain ordering and constraints are provided below with respect to FIG. 14. In this way, the user may enter phrases that are more natural and without having to specify the specific clinical markers or key words that may define an event of interest. In cases of long cycle cancers such as breast cancer, acute myeloid leukemia (AML), acute lymphocytic leukemia (ALL) and multiple myeloma (MM), key index dates are important to track to guide treatment decisions (diagnosis date, treatment start date, last visit, last adverse event (AE), etc.). This reduces the need to search for a specific event manually and can show the user the most relevant or desired time frame. This applies to clinical trials where the references to AEs or trial compliance related events are being tracked.

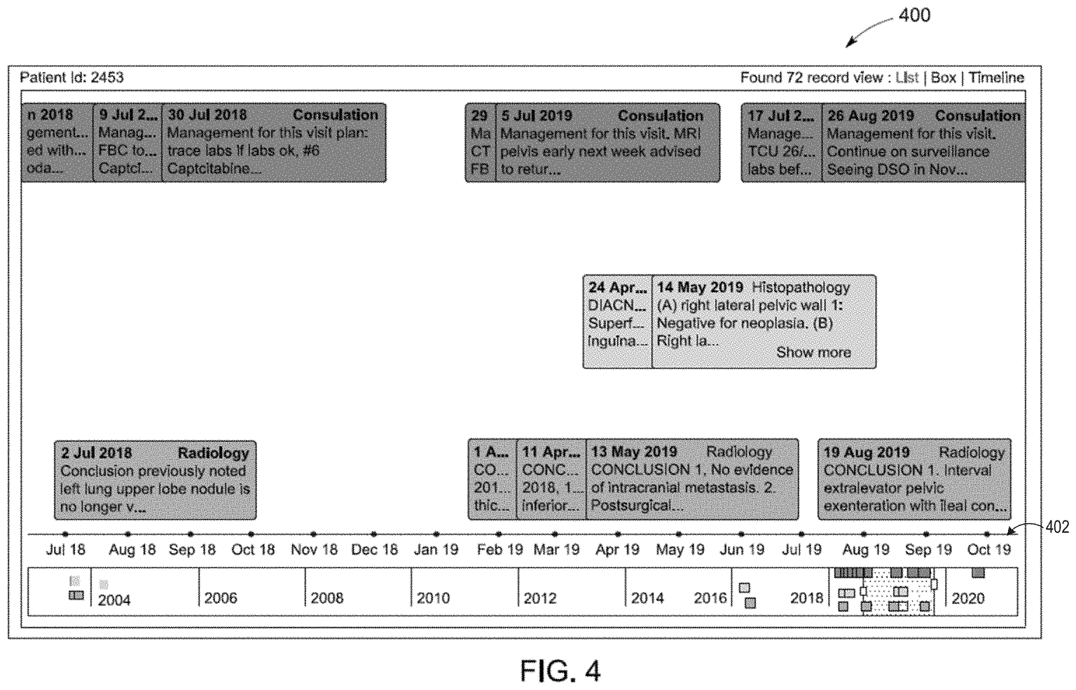

An example of a timeline segment 400 generated via natural temporal searching is shown in FIG. 4. The identified events are shown in a timeline format (time-ordered) with different categories of events or records positioned into different swimlanes. For example, the segment 400 shown in FIG. 4 includes a consultation swimlane, a histopathology swimlane, and a radiology swimlane, though other swimlanes are possible without departing from the scope of this disclosure. A longer timeline 402 may be shown across a bottom of the timeline segment, with the current timeline segment (e.g., from July 2018 through August 2019) shown by highlighting. Thus, an NLP-based search may return structured table-like data which can be plotted on a timeline. If the clinician wants to see only a semantic subset (say “only abdominal ultrasounds” or “only urology issues”) it is possible to sub-select for the requested semantic subset easily. This format may be easier to analyze and find relevant information than conventional search returns that provide a list of documents with snippets of text.

By doing so, AEs and key events may be identified to reduce cognitive loads, and create custom cancer journey reports for a specific need. The timeline can navigate to the specific time point without searching through a large set of data. This also helps in reducing the visual dimension of the long cycle cancers (searching for a data point even when it is not visible within the screen size).

In the diagnosis, treatment, and management of certain patient conditions such as cancer, many different types of clinicians may be involved in the decision making and care delivery for the patient, including oncologists, nurses, radiologists, pathologists, surgeons, and so on. As such, a general patient timeline may not be optimal for each clinician, as some information may not be relevant to that clinician. Having to navigate through the timeline and all associated data to find information of interest may be time-consuming and difficult. Thus, the display of a timeline may be customized based on a user's specialization.

The customization may include adding or removing elements of the timeline based on the specialization of the user who is viewing the timeline currently. Thus, the displayed patient information timeline includes elements of the patient history and data which are used for the completion of a specific task(s) a given clinician is to perform or to follow up with the patient. The set of data elements/details would be a combination of extracted data from various systems including processed data through NLP/AI technologies. Such details would be configurable at institutional or at individual user levels as appropriate.

For example, if the user is a surgeon planning a surgery on the patient, the subset of information which is relevant for the surgeon to plan the surgery is chosen. For example, the timeline may be adjusted to include the spatial location of the tumor(s), size of each tumor, type of each tumor, margin length of each tumor, lymph-nodes which are involved, and co-morbidities of the patient. The level of details needed for each swimlane and tuple of the timeline are different for each care team member. For example, if a pathologist is logged in (e.g., the user specialization is for a pathologist), the default level of the timeline will show more details of pathology and lab tests. Likewise, a radiologist will see more details on the radiology swimlane. This adaptation of the swimlanes and the level of details greatly reduces the cognitive load, and allows each care team member to focus on their specific context. This timeline customization may be expanded to include timeline customization based on stage of the disease, current treatment, and so on. In one example, a representation method may be applied to capture the context and then use ontology to map the relevancy of each information to the context.

As appreciated from FIGS. 1-4, the timelines disclosed herein are populated with information from patient EMRs, pathology reports, biomarker reports, imaging exam reports, each of which include findings, summaries of discussions, etc., documented by a clinician. As such, the amount and quality of the information included in and/or linked to in the timeline is based on the quality of the report/record generation by each clinician. To facilitate enhanced report/record generation, an artificial intelligence (AI) assisted method may be applied by the presentation system 102 to review and prompt a clinician for reporting on relevant data elements as a continuum of the prior tracked parameters, and highlight the gaps.

To facilitate this, the presentation system 102 may present a minimum set of parameters being tracked longitudinally for a patient. The minimum set of parameters may be based on the type of the report (e.g., consultation, radiology, pathology, etc.), and the diagnostic purpose of the report in the context of the current stage of treatment (e.g., risk assessment, pre-treatment evaluation, etc.). For example, if the report is created after a consultation for lung cancer risk assessment, the minimum set of parameters as required by the lung cancer treatment guideline may include age, smoking history, previous cancer history, occupational exposures, other lung diseases, etc. This information is collected from the longitudinal data of the patient. The presentation system 102 also makes use of a database of high priority variables to track the variables. High priority variables may include variables which need to be tracked continually throughout the patient's cancer treatment and monitoring progression. For example, the high priority variables may include tumor locations, tumor types, tumor sizes, and primary vs secondary tumor. This database can be created by clinicians as well as created automatically from care guidelines. Written documents (e.g., reports and records) may be tracked and suggestions may be provided to reflect the remaining items. In some examples, standard templates for radiology/pathology reports may be created. In some examples, the parameters that are tracked may be determined by doing user research, combining them with the knowledge of guidelines and key clinical trials that are being pursued in the industry. The same will be enhanced by working with researchers to advance and refine the same to produce new knowledge and scaling of the same from academic centers to community centers.

Thus, a report generation model may be deployed to provide suggestions to clinicians for information to be included while generating patient reports and/or provide templates that may guide the clinicians in the report generation to ensure target information is included in each report. To accomplish this, the report generation model may evaluate the patient's current path as to condition diagnosis, treatment, monitoring, and outcomes based on the patient's longitudinal medical data (e.g., the patient's digital twin as described in FIG. 1). The patient's path may be compared to selected guidelines for treating the patient condition to identify the parameters that should be tracked for that patient, such that parameters relevant to the guidelines are tracked. Once the parameters have been identified, the parameters may be output to the clinician during report generation and/or a report template may be generated with each parameter included in the template, so that the clinician can fill in the patient specific values/information for each parameter. In some examples, the patient's path may be compared to a cohort of similar patients, and the report generation model may identify the parameters that were tracked for the patients in the cohort. The report suggestions and/or template may be generated based on the parameters tracked in the cohort. In doing so, the quality and completeness and continuum of reports may be improved. Further, the patient may be monitored in close proximity with the guidelines, by forcing the clinicians to report on these elements.

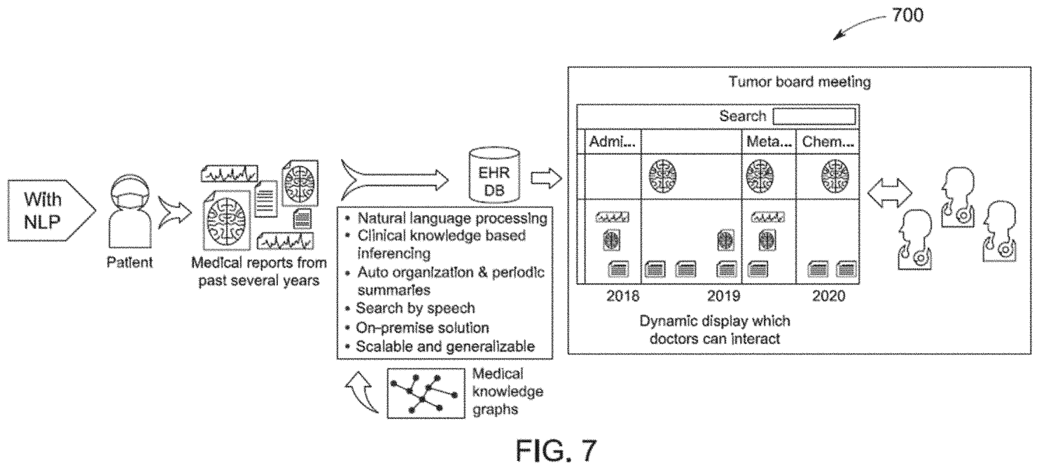

The combination of the longitudinal patient information presentation and the natural language processing may provide several benefits. In the context of managing cancer treatment and as depicted visually by process 700 of FIG. 7, a patient's medical reports from a segment in time (e.g., past several years) may be aggregated and analyzed using NLP. Clinical knowledge based inferencing may be applied. Auto-organization may be performed and periodic summaries may be generated. The NLP may provide for search by speech. The presentation system 102 described herein may be an on-premise solution that is scalable and generalizable. By doing so, clinical staff time per patient may be reduced, manual errors may be reduced, time periods and disease progression may be visualized, exploration and discovery may be enabled, and speech-based navigation of patient history may be provided.

Further, clinicians may have many areas where current data access protocols via standard EMRs, pathology reports, imaging reports, etc., fall short, resulting in wasted time and effort on the part of the clinicians. For example, clinicians may desire to view all relevant data for a patient in one location, rather than having to hunt and navigate through multiple interfaces to find the desired data. Clinicians may desire to get a big picture view, and then drill down to more detailed views from the big picture views. Clinicians may desire to quickly navigate to desired data, see overall trends in patient condition, and compare a current patient with a cohort of patients. With the current siloing of medical data into different databases/storage systems with different communication protocols and data formats, clinicians may interact with separate interfaces and view multiple pieces of patient data to assemble a complete desired dataset. Performing searches for desired data may be difficult and require knowledge of what search parameters to use for each different data system/interface. For example, a search for DICOM data may necessitate queries in a first format while a search for pathology data may necessitate queries in a second, different format. All told, tracking and comprehending the current status of a patient is time-consuming and places a large mental load on clinicians. This process is also inefficient from a processing and network data standpoint, as it may result in more searches being performed than necessary, retrieval of undesired information, prolonged display of various menus, etc., which may waste processing resources and increase network traffic.

The longitudinal presentation system described herein may alleviate these issues by aggregating data from multiple repositories to a single view (e.g., the timeline disclosed herein) in a single browser, aggregating data from multiple applications and systems to a single view (e.g., the timeline disclosed herein) in a sorted manner, extracting and transforming scattered data into key data elements from multiple reports into a single view (e.g., the timeline disclosed herein), including radiology, endoscopy, pathology dates, types, and key results presented as a big picture view. Further, diagnostic workup, treatment plans, multi-disciplinary team (MDT) notes, and dates are visualized in a time sorted order on their axes on the timeline. Different treatment types —chemo, surgery, radiation, immune, hormonal, patient ECOG—may be trended over time. Searches may be performed with patient parameters, disease state, and attributes for a listing across the medical facility, using natural language and not requiring specific search query formats. Patient events, toxicities, show symptoms, ECOG status, PROs, and encounters may be summarized and time sorted. The timeline may be scrolled to focus on a previous encounter, and/or a default view may be chosen to show previous encounter. Tumor parameters may be trended with a single click with extracted radiology/pathology/biomarkers.

The timelines disclosed herein may be updated in a clinician specific manner (e.g., based on the clinician's specialty), and also in a patient-condition specific manner. For example, the timelines may be adjusted based on whether the patient has lung cancer, breast cancer, prostate cancer, etc., so that the information most relevant to each different type of cancer is presented. When desired or appropriate, guidelines for treating and monitoring each cancer may be integrated into the timeline, to facilitate fast and easy evaluation of the patient's treatment and progression relative to the standard of care. When deviations are present, the differences between the patient's treatment relative to the guidelines may be highlighted. Similarly, the patient may be compared to other patients and a cohort of similar patients may be identified. Summaries of the patients in the cohort may be provided on the timeline (e.g., that highlight similarities and differences between the patient and the cohort), as well as suggestions for treatment, parameter evaluation, etc., that are based on the cohort. Further, patient biomarkers such as genomics may be integrated into the timeline. In addition to including genomic reports in the timeline, predictions for treatments or treatment response based on a patient's individual genomics may be provided via the timeline and presentation system disclosed herein.

Thus, the presentation system disclosed herein may provide a view of a patient's journey in the form of a timeline that incorporates information from the patient's EMR as well as integrating pathology reports, imaging, genomic reports, etc. The timelines may be presented in a cancer-specific manner, e.g., specific for lung cancer, prostate cancer, breast cancer, and so on. The presentation system may leverage NLP to provide smart searching. The timelines may be exported to the patient's EMR and be accessible to all clinicians on the patient's multi-disciplinary team (MDT). The presentation system may import treatment guidelines and integrate the guidelines into or on the timeline display. The presentation system may utilize similar patient cohorts with integrated imaging and genomics to highlight similarities and differences between the patient's journey and that of the cohort. Treatment response prediction for cancer may be provided based on the patient's genomic reports and/or radiomics. The presentation system may obtain external data, such as from cancer registries, and present the information when appropriate to clinicians via the timeline. The presentation system may provide multi-EMR compatibility, integrate imaging and text, and provide care pathway metrics. The above features may be facilitated by leveraging a variety of technologies, including data aggregation, NLP (e.g., NLP for reports, NLP for consults), NLP polyglots, AI, summarization, scaling on the cloud, bi-directional smart EMR adapters, historical data processing, multi-modal clinical decision support (CDS) (e.g., image, PGHD, and text decision systems; recommendation and predictor systems), integration with existing applications, and integration with third party solutions. In doing so, clinician cognitive load may be reduced and patient care may be improved. Further, processing resources of one or more computing devices may be utilized more efficiently and network traffic may be reduced by reducing clinician searches and interactions with multiple different interfaces.

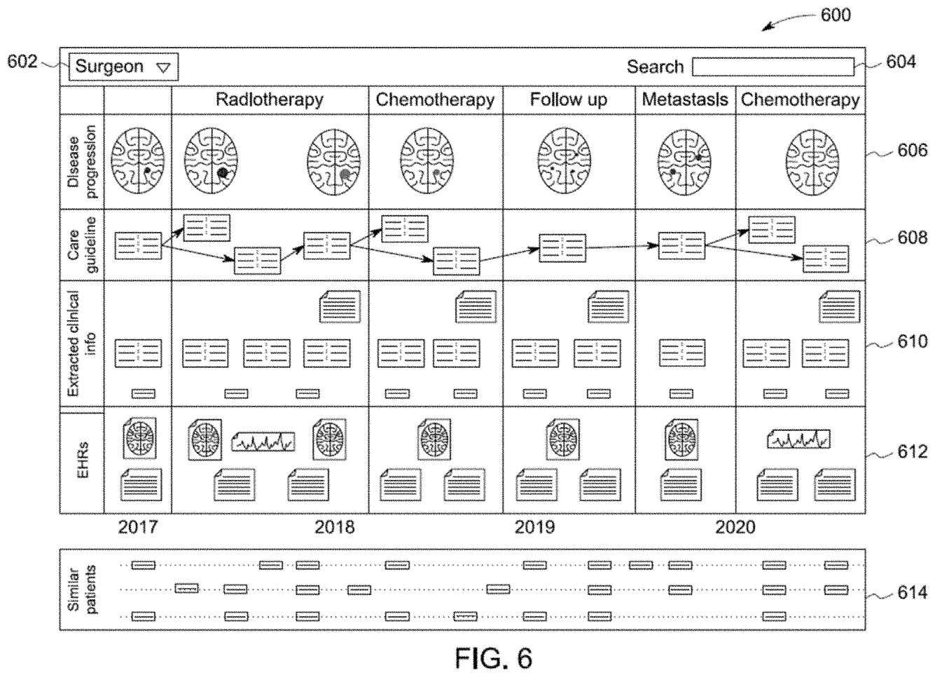

FIG. 6 shows another example timeline 600. As shown in FIG. 6, a drop-down menu 602 may be included where a clinician specialty may be selected (e.g., surgeon, radiologist, etc.). When a specialty is selected, the information included on the timeline may be adjusted as explained above. Also shown in FIG. 6 is a search bar 604, in which a user may enter natural language search queries (e.g., metastasis phase), as explained above. In FIG. 6, disease progression and identification may be visualized, as shown by the images in section 606 that schematically depict an anatomical region of interest (e.g., a brain) and tumor progression for one or more tumors identified in the anatomical region of interest. When desired, care guidelines applicable to the patient may be identified, extracted, and included as an overlay, shown in section 608. Further, extracted clinical information which may include multi-report/record summaries, structured data and trend/anomaly information, and per-report summaries may be generated and included on the timeline, as shown in section 610. In section 612, related resources and/or patient EMRs may be illustrated.

Section 614 shows how identified similar patients (also referred to as reference patients) may be depicted as part of the timeline. A summary may be generated for each reference patient, highlighting the similarities and differences in the journeys between the patient and the reference patients.

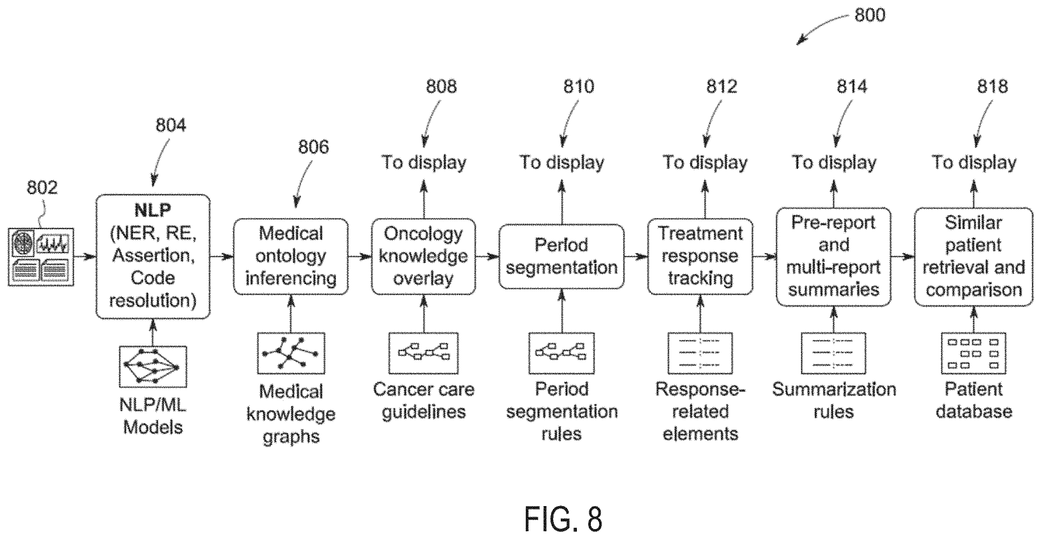

FIG. 8 provides an example overview 800 of how presentation system 102 may be utilized to generate timelines and associated display elements that may aid in delivering patient care. The overview 800 may represent a method for generating timelines that may be executed according to instructions stored in memory of a computing device, e.g., the presentation system 102 of FIG. 1. As shown in FIG. 8, the presentation system 102 may ingest patient data 802, as described above with respect to FIG. 1. The patient data may be in multiple different formats and obtained from different sources. The presentation system 102 may utilize NLP module 126 to perform NLP on the patient data, as shown at 804. The NLP may include named entity recognition (NER), entity resolution, assertion, code resolution, and so on, as described above. Medical ontology inferencing may be performed on the processed data at 806, utilizing medical knowledge graphs. In this way, relevant clinical markers and information in the medical data may be identified and extracted, which may then be used for downstream display elements/overlays, as explained herein. For example, at 808, the processed patient data may be used to generate an oncology knowledge overlay based on cancer care guidelines, so that the guidelines relevant to the current patient status/condition may be displayed. At 810, period segmentation may be performed on the processed patient data according to period segmentation rules, and the period segmentation may be used to time order and segment the events of the patient data for display in the timeline. At 812, treatment response tracking may be performed on the patient data using response related elements, and any identified treatment responses may be displayed. Response related elements may include entities specific to treatment response. For example, in cancer, response elements may include “stable response,” “no response,” etc., which may be determined following response evaluation criteria specific to a given condition or treatment, which allows clinicians to know if a treatment is working. At 814, per-report and multi-report summaries may be generated based on the relevant/extracted patient data (including treatment response) and summarization rules, and the summaries may be displayed on the timeline. At 818, the patient data may be used to identify similar patients from a patient database, and information about the similar patients may be retrieved and used to generate comparison summaries or other information that may be displayed.

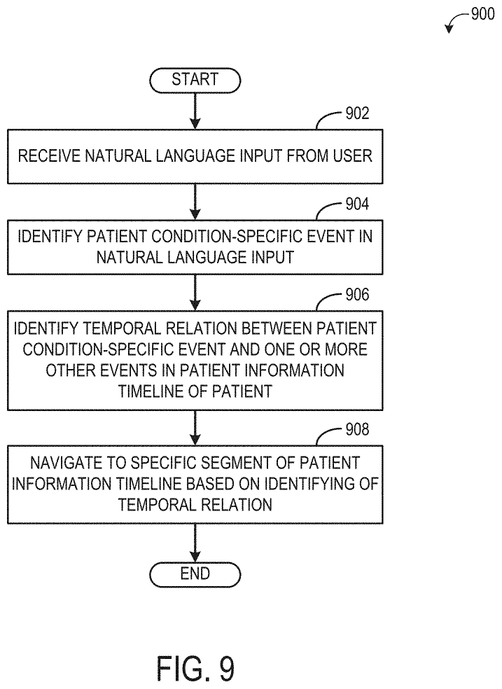

FIG. 9 illustrates a method 900 for identifying and navigating to a segment of a patient information timeline which includes events related to a natural language query. The method 900 may be carried out according to instructions stored in memory of a computing device, such as the presentation system 102, to help the user navigate to an appropriate time-point in the patient information timeline using temporal event-related phrases.

At 902, the method 900 includes receiving a natural language input from a user. For example, a clinician may enter a word or phrase (via a suitable user input mechanism) which includes keywords associated with a medical condition. The NLP module may analyze voice communication and/or text input to obtain and/or infer various information related to the patient history, clinical queries, and so on. As described with respect to FIG. 1, the presentation system 102 and/or the care provider device 134 may include a microphone used to receive the natural language input. In some examples, the natural language input may be received via text input (e.g., a keyboard, touchscreen, etc.).

At 904, the method 900 includes identifying a patient condition-specific event in the natural language input. The NLP may include named entity recognition (NER), entity resolution, assertion, code resolution, and so on, which may be used to determine a patient condition-specific event (e.g., a disease stage, a procedure, a treatment, and so on).

At 906, the method 900 includes identifying a temporal relation between the patient condition-specific event (e.g., identified at operation 904) and one or more other events in the patient information timeline of the patient. For example, the one or more other events may be procedures, treatments, and so on which were performed as part of a treatment pathway for the identified patient condition-specific event (e.g., a diagnosis).

At 908, the method 900 includes navigating to a specific segment of the patient information timeline based on the identified temporal relations (e.g., between the patient condition-specific event and the one or more other events). For example, the specific segment of the patient information timeline may start at the patient condition-specific event and extend temporally until the patient condition-specific event is identified as being resolved. In other examples, the specific segment may include relevant events leading up to the patient condition-specific event. The specific segment may be displayed on a display device, for example, as shown in FIG. 4. In this way, a clinician may be able to navigate to a desired segment of a timeline using natural language inputs without knowing in advance which particular terminology was used in the reports. As an example, a clinician may ask to navigate to a segment of a timeline showing “first line treatment,” but no reports may use that term. Standard keyword searching would show no results for such as search. The NLP-based searching described herein can infer the meaning of the search term in medical ontology (when a first treatment for a condition was administered) and can navigate to that segment of the timeline.

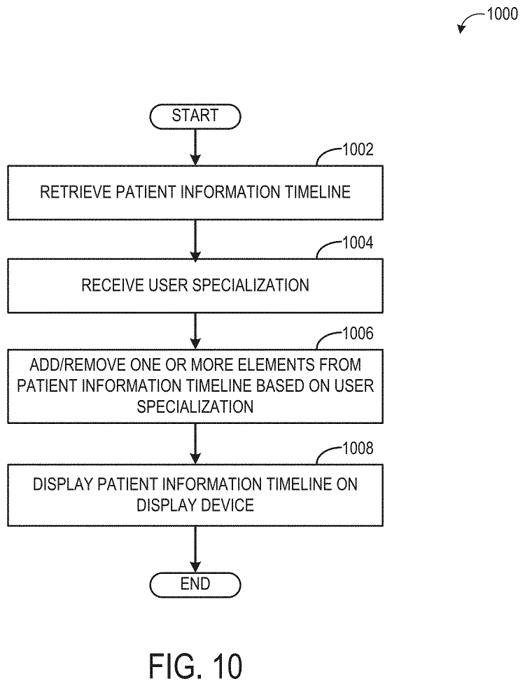

FIG. 10 illustrates a method 1000 for modifying a patient information timeline based on a user's specialization. The method 1000 may be carried out according to instructions stored in memory of a computing device, such as the presentation system 102, and may modify a patient information timeline generated as described herein by the presentation system 102.

At 1002, the method 1000 includes retrieving a patient information timeline. In some embodiments, the patient information timeline may have been previously generated and may be stored in memory of the presentation system 102. In other embodiments, the patient information timeline may be generated at operation 1002 according to the methods described herein for generating a patient information timeline (e.g., the process described above with respect to FIG. 8). The patient information timeline includes a plurality of elements, where each of the plurality of elements visually represents a patient condition-specific medical event, record, and/or report, and the plurality of events are displayed in a time-ordered fashion.

At 1004, the method 1000 includes receiving a user specialization. For example, when turning on or otherwise activating the presentation system 102, a user may input credentials which include a specialization of the user, such as surgeon, anesthesiologist, radiologist, and so on. Additionally or alternatively, a specialization may be selected from a drop-down menu or other list of specializations on a display device/user interface, as is shown in FIG. 6.

At 1006, the method 1000 includes adding or removing one or more elements of the plurality of elements from the patient information timeline based on the user specialization. For example, information which is relevant to the selected specialization may be included on the patient information timeline and information which is not relevant may not be included on the patient information timeline. As an example, a dietician may see complications related to diet (e.g., vomiting, weight) and may not see complications related to the heart; a nephrologist will see complications related to the kidney.

At 1008, the method 1000 includes outputting the patient information timeline for display on a display device. The patient information timeline may be modified from its originally generated form to exclude events which may not be relevant to the selected user specification.

The presentation system 102 described herein may generate timelines for patients and may be particularly beneficial for long-term conditions such as cancer. Cancer is frequently treated via chemotherapy, where various chemical agents may be provided to a patient to selectively kill or inhibit growth of tumor cells. While chemotherapy is generally administered in a hospital or other medical facility, the cost associated with traditional chemotherapy is high and in some circumstances, this cost may be lowered by providing chemotherapy at the patient's home. However, such at-home infusions may carry risks if the patient lives far from a medical facility that could provide assistance in the event of an adverse event.

As explained above with respect to FIG. 1, the presentation system 102 may be in communication with a plurality of different medical/patient databases that each store different types of medical data of patients. The presentation system 102 may analyze these different medical data to identify and combine various risk factors of the patient to decide if the patient is suitable for home-infusion. The presentation system 102 may obtain values of certain parameters of the patient in the patient's medical data and compare them to a reference database to compute a risk score.

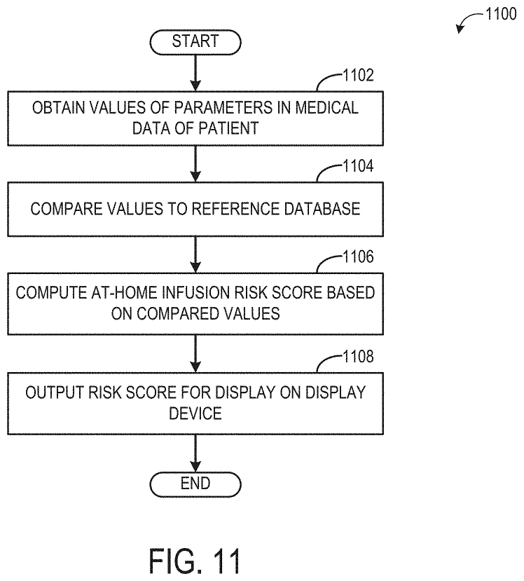

FIG. 11 illustrates a method 1100 for analyzing patient medical data and calculating an at-home infusion risk score based on the patient medical data. The method 1100 may be carried out according to instructions stored in memory of a computing device, such as the presentation system 102, and may acquire medical data from databases coupled to the presentation system 102, such as the PACS 110, the RIS 112, the EMR database 114, the pathology 116, and the genome 118.

At 1102, the method 1100 includes obtaining values of certain parameters in medical data of a patient. A list of the certain parameters may be sourced from a configuration database. For example, the list of parameters to track are read from a configuration database. The list of parameters may include different types of parameters which can impact the suitability for home infusion (such as distance to the nearest hospital, frequency of nurse-visits, etc.).

At 1104, the method 1100 includes comparing the values to a reference database (e.g., the configuration database). Values of the reference database may include desired parameter values and/or values of a healthy patient (e.g., without pathology).

At 1106, the method 1100 includes computing an at-home infusion risk score based on the comparing (e.g., the compared obtained parameter values and the reference database). The risk score represents a predicted level of risk for at-home infusion of chemotherapy for the patient. A combined risk-score may be calculated by weighing individual risk-scores by a certain weight vector and adding them together to get a final risk score. The weight vector is also read from the configuration database.

In some examples, the risk for adverse events for home infusions may be predicted by combining a disease model, a drug model, and a patient co-morbidity model. The disease model may generate a first risk score for the patient based on the type of cancer the patient has, for example. The drug model may generate a second risk score for the patient based on the type(s) of drug(s) being administered to the patient via the chemotherapy. The co-morbidity model may generate a third risk score for the patient based on the patient's co-morbidities. Each risk score may reflect a likelihood that the patient may undergo an adverse event while receiving chemotherapy. Other models may also be included, such as a biomarker model that generates a fourth risk score based on patient biomarkers (e.g., tumor genotype, tumor proteins). Each individual risk score may be weighted and then combined to generate the final risk score. The final risk score may further include a mitigating factors risk score, which may reflect the patient's ability to receive treatment in the event that an adverse event does occur. The mitigating factors risk score may be based on the patient's distance to a medical facility, availability and type of treatment required for the adverse event(s) predicted for the patient, average outcomes of the predicted adverse event, and so on. The risk scores may be calculated from simple progression of disease (like doubling time of tumor). The risk scores could be calculated from various parameters from patient reported outcomes to the activity levels and/or a combination of above. The risk scores may also be generated by generating data and deploying an AI algorithm leveraging patient vital signs from a home monitoring unit and combining the vital signs with EMR data and the various patient generated outcome data.

At 1108, the method 1100 includes outputting the final risk score for display on a display device. If the combined risk-score (e.g., the final risk score) meets a condition relative to a threshold set in the configuration database, the patient may be deemed suitable for at-home infusion.

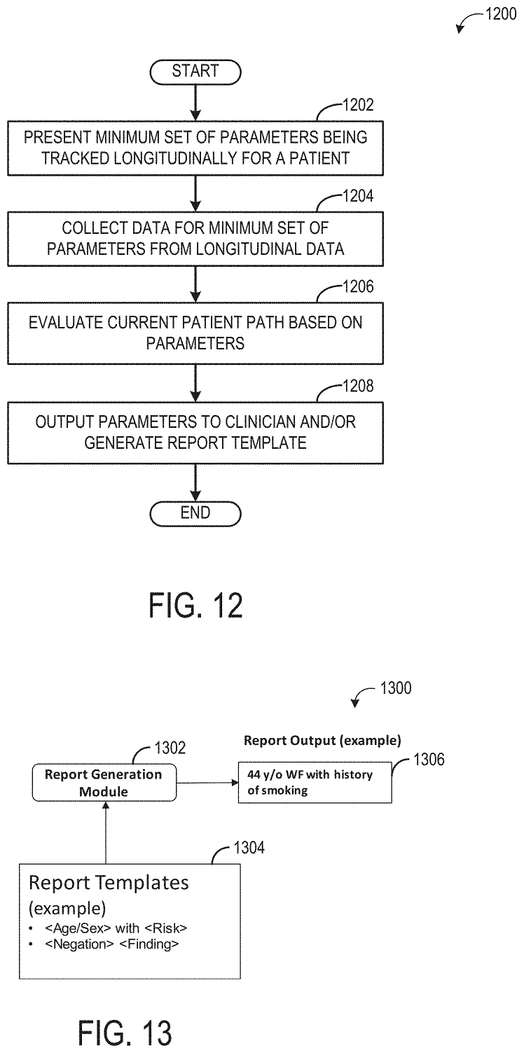

FIG. 12 illustrates a method 1200 for facilitating enhanced report and/or record generation. The method 1200 may be carried out according to instructions stored in memory of a computing device, for example method 1200 may be implemented using the report generation model 127 of the presentation system 102 and may acquire medical data from databases coupled to the presentation system 102, such as the PACS 110, the RIS 112, the EMR database 114, the pathology 116, and the genome 118. Method 1200 may be executed in response to a determination that a report for a patient is being generated (or about to be generated), which may include determining that a user input has been received indicating that a report is to be generated.

At 1202, the method 1200 includes presenting a minimum set of parameters which are being tracked longitudinally for a patient. For example, the minimum set of parameters may be based on the type of the report (e.g., consultation, radiology, pathology, etc.), and the diagnostic purpose of the report in the context of the current stage of treatment (e.g., risk assessment, pre-treatment evaluation, etc.). For example, if the report is created after a consultation for lung cancer risk assessment, the minimum set of parameters as required by the lung cancer treatment guideline may include age, smoking history, previous cancer history, occupational exposures, other lung diseases, and so on. This information is collected from the longitudinal data of the patient.

At 1204, the method 1200 includes collecting data for the minimum set of parameters. For example, collecting data may include retrieving information from longitudinal patient data (e.g., as shown in a patient information timeline), a database of high priority variables, written documents, and so on. High priority variables may include variables which need to be tracked continually throughout the patient's cancer treatment and monitoring progression. For example, the high priority variables may include tumor locations, tumor types, tumor sizes, and primary vs secondary tumor. This database can be created by clinicians as well as created automatically from care guidelines. Written documents (e.g., reports and records) may be tracked and suggestions may be provided to reflect the remaining items. In some examples, standard templates for radiology/pathology reports may be created. In some examples, the parameters that are tracked may be determined by doing user research, combining them with the knowledge of guidelines and key clinical trials that are being pursued in the industry. The same will be enhanced by working with researchers to advance and refine the same to produce new knowledge and scaling of the same from academic centers to community centers.

At 1206, the method 1200 includes evaluating a current patient path based on collected data for the minimum set of parameters. Evaluating the current patient path may include deploying a report generation model to provide suggestions to clinicians for information to be included while generating patient reports and/or provide templates that may guide the clinicians in the report generation to ensure target information is included in each report. To accomplish this, the report generation model may evaluate the patient's current path as to condition diagnosis, treatment, monitoring, and outcomes based on the patient's longitudinal medical data (e.g., the patient's digital twin as described in FIG. 1). The patient's path may be compared to selected guidelines for treating the patient condition to identify the parameters that should be tracked for that patient, such that parameters relevant to the guidelines are tracked.

At 1208, the method 1200 includes outputting identified parameters to the clinician during report generation and/or generating a report template with each parameter included in the template, so that the clinician can fill in the patient specific values/information for each parameter. In some examples, the patient's path may be compared to a cohort of similar patients, and the report generation model may identify the parameters that were tracked for the patients in the cohort. The report suggestions and/or template may be generated based on the parameters tracked in the cohort. In doing so, the quality and completeness and continuum of reports may be improved. Further, the patient may be monitored in close proximity with the guidelines, by forcing the clinicians to report on these elements.

FIG. 13 shows an example process 1300 for generating a report using a report generation model 1302, which is a non-limiting example of report generation model 127 of FIG. 1. The report generation model 1302 may generate or obtain a report template, such as report template 1304, that specifies the information to be included in the report, the order the information should be included in the report, and other formatting features. For example, report template 1304 may specify that a patient's age and sex be included in the report along with one or more risk factors. The report generation model 1302 (or a clinician) may output a report 1306 by filling in the template with the specified information, e.g., the report 1306 may state that the patient is a 44-year-old female with a history of smoking.

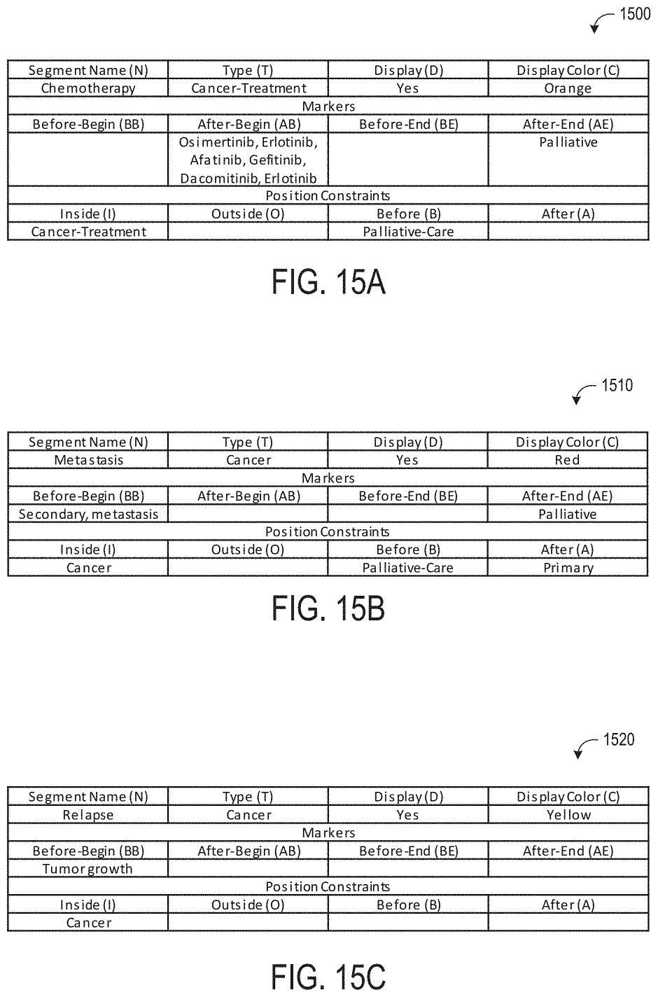

FIG. 14 shows an example table 1400 of domain ordering constraints that may be applied to temporally order reports/timeline entries, resolve ambiguous entry boundaries, facilitate timeline searching, and the like. Table 1400 may store time-segment information, markers, and position constraints for a segment of timeline entries. The segment may include one or more timeline entries where the one or more timeline entries share a common time frame (e.g., a month or a year) or include a common entity (e.g., disease, anatomy, treatment). A similar table may be generated and stored for each of a plurality of different segments. Table 1400 and other segment tables may be applied when generating or searching timelines for one or more patients. The rules/relationships specified by the tables may not be patient specific, but the tables (e.g., table 1400) may be populated with patient-specific information when generating a timeline for a specific patient. Example populated tables are shown in FIGS. 15A-15C and described in more detail below.

A segment name field 1402 of table 1400 may specify a unique name or ID of a segment. For example, the segment field may specify that the table 1400 applies to timeline segments related to chemotherapy, a particular stage of cancer (e.g., metastasis), or another suitable type of segment. A type field 1404 may reference to segment types which have been created already, which may help reuse position constraints. For example, if the segment field specifies the segment is chemotherapy, the type field 1404 may specify that the segment is a cancer treatment. When the type field is populated, position constraints from a previously created timeline segment of the same type may be filled or used to determine the position constraints of the current table. A display field 1406 may be filled to specify whether or not the segment is or will be displayed, which allows for specification of invisible segments for internal book-keeping, which helps simplify segment definitions. A color field 1408 defines the display color of the segment.

Table 1400 may order clinical markers of the segment (extracted from a sequence of EMRs, as shown schematically at 1410) based on a temporal relationship of the markers to the segment. For example, table 1400 includes a set of markers fields 1412, which in the example shown herein includes four fields for specifying the temporal nature of the clinical markers: before-begin (BB), after-begin (AB), before-end (BE), and after-end (AE). Example clinical markers include tumor stage, complications, treatments, etc. Again using chemotherapy as an example segment, chemotherapy agents that are administered to a patient may be specified in the AB field (as the agents are administered after chemotherapy has begun).

Table 1400 also specifies position (e.g., timing) constraints of the segment relative to other events/segments. Table 1400 includes a set of position constraints fields 1414. The position constraints specified by table 1400 include inside, outside, before, and after. Using chemotherapy as an example segment, chemotherapy may be administered as a cancer treatment, and thus falls “inside” a cancer treatment event. In contrast, chemotherapy may occur before a remission event. Thus, for chemotherapy, cancer treatment may be populated in the inside field and remission may be populated in the before field.

The information stored in table 1400 or other similar tables may be used to resolve ambiguous segment boundaries. For example, as shown schematically by process 1420, a set of segments 1422 (each of which may include one or more timeline entries) may be ordered temporally (e.g., with time increasing from left to right) and by event (e.g., with different swimlane categories extending from top to bottom). As shown within the dotted circle, some of the segment boundaries may be ambiguous, such as segment 1424, which overlaps two other segments (e.g., overlapping temporally with a first adjacent segment and event-based with a second adjacent segment). Segment 1424 may have ambiguous boundaries because it may not be clear from the EMRs when the segment ended. For example, if segment 1424 is chemotherapy, the EMRs may not explicitly state that chemotherapy was stopped on a particular date.

However, by applying the constraints specified by table 1400, the ambiguous boundaries may be resolved, as shown by the resolved set of segments 1426. For example, segment 1424 may be adjusted so that the segment ends when the first adjacent segment begins. The constraints applied to resolve this ambiguity may include determining that the chemotherapy ended on a particular date, as the patient was moved to palliative care on that particular date.

FIG. 15A shows a first example table 1500 that illustrates the ordering relationship for the segment “chemotherapy” and a particular chemotherapy agent (e.g., Osimertinib) for a patient. The chemotherapy segment is specified as being a cancer treatment type of segment that is displayed using the color orange, though the color is for illustrative purposes and could be any suitable color. The chemotherapy agent is a clinical marker that occurs after the beginning of chemotherapy (along with additional chemotherapy agents) and thus is listed in the AB field of the set of markers fields. Another cancer treatment, palliative care, is also shown in table 1500, which is a clinical marker that occurs after chemotherapy ends and thus is listed in the AE field. As explained previously, chemotherapy is a cancer treatment and thus chemotherapy occurs inside cancer treatment, which is thereby listed in the inside field of the set of position constraints fields. Chemotherapy occurs before palliative care and thus palliative care is listed in the before field of the set of position constraints fields.

FIG. 15B shows a second example table 1510 that illustrates the ordering relationship for the segment “metastasis” and the clinical marker “secondary tumor” for a patient. The metastasis segment is specified as being a cancer type of segment that is displayed with the color red. The clinical markers “secondary,” “metastasis,” and “palliative” are shown in the set of markers fields, with both secondary and metastasis occurring before the metastasis segment and thus listed in the BB field, while palliative care occurs after metastasis ends and thus is listed in the AE field. Metastasis occurs within the disease cancer, and thus cancer is listed in the inside field of the set of position constraints fields. Palliative care is listed in the before field, as metastasis occurs before palliative care. Primary is listed in the after field, as metastasis occurs after the primary tumor.

FIG. 15C shows a third example table 1520 that illustrates the ordering relationship for the segment “relapse” and the clinical marker “tumor growth” for a patient. The relapse segment is specified as being a cancer type of segment that is displayed with the color yellow. The clinical marker “tumor growth” is shown in the set of markers fields, with tumor growth occurring before the relapse segment and thus listed in the BB field. Relapse occurs within the disease cancer, and thus cancer is listed in the inside field of the set of position constraints fields.

The technical effect of presenting patient timelines as described herein is that multiple years of reports (e.g., EMRs), which may amount to hundreds of reports, may be displayed in a condensed manner that allows clinicians to easily search and find specific reports. Specifically, the reports are represented by small snippets of relevant text and/or by symbols (referred to as entries) and the entries are divided into lanes by category (e.g., pathology, radiology, etc.) ordered temporally, which provides an improvement to the capability of a healthcare system as a whole. The disclosure provides a specific way of improving the capability of the healthcare system, by providing one or more timelines that display dynamically updating patient medical events/records in a longitudinal manner. The disclosure further provides a specific improvement to the way computers operate by aggregating patient medical information for multiple separate databases/data storage systems in one location and updating the timelines in real-time and as demanded, which may obviate the need for users to have to navigate through multiple different data files/system interfaces, perform cumbersome and unnecessary searches that may not return relevant results, and so forth, thereby increasing the efficiency of the operation of the computer for the user.

The timelines described herein provide a specific manner of displaying a limited set of information to a user (patient medical information), rather than using conventional user interface methods to display a generic index on a computer, requiring the user to step through many layers of menu options to reach the desired data, or burying the desired data within scores of less relevant, routine patient records. Thus, the user experience with the computer may be improved and made more efficient.

Furthermore, by displaying a limited set of information via the timelines as described herein, operation of the computing device(s) that collect and render the data for display may be improved by reducing the processing demands of the computing device(s), thereby increasing the efficiency of the computing device(s). For example, only certain patient medical records may be displayed or only certain information from each patient medical record may be displayed, which results in a limited amount of the data that is received being processed, which may improve the efficiency of the computing device(s).

In another representation, a method includes obtaining values of certain parameters in medical data of a patient, comparing the values to a reference database, computing an at-home infusion risk score based on the comparing, the risk score representing a predicted level of risk for at-home infusion of chemotherapy for the patient, and outputting the risk score for display on a display device.

In another representation, a computing device comprises a display screen, the computing device being configured to display on the screen a timeline listing one or more patient medical events obtained from one or more patient data sources, and additionally being configured to display on the screen a details panel that can be reached directly from the timeline, wherein the details panel displays a limited list of data offered within the one or more patient data sources, one or more of the data in the list being selectable to launch an interface associated with the respective data source and enable the selected data to be seen within the interface, and wherein the details panel is displayed while the one or more data sources are in an un-launched state.

The disclosure also provides support for a computing device comprising a display screen, the computing device being configured to display on the screen a timeline of patient medical information including a plurality of symbols representing the patient medical information, wherein a symbol of the plurality of symbols is selectable to launch a details panel and enable a report that references the displayed patient medical information to be seen within the timeline, and wherein the symbol is displayed while the details panel is in an un-launched state. In a first example of the computing device, the plurality of symbols is displayed in one or more rows, each row corresponding to a different category of patient medical information, and the symbols in each row are ordered by time. In a second example of the computing device, optionally including the first example, each symbol of the plurality of symbols represents a patient medical event, a patient medical report, or patient medical data identified from one or more patient data sources. In a third example of the computing device, optionally including one or both of the first and second examples, the details panel includes a summary of information included in the report. In a fourth example of the computing device, optionally including one or more or each of the first through third examples, the patient medical information represented by the plurality of symbols relates to a specific patient medical condition and is originally stored in a plurality of separate data sources. In a fifth example of the computing device, optionally including one or more or each of the first through fourth examples, the plurality of separate data sources comprises two or more of a picture archiving and communication system, a radiology information system, an electronic medical record database, a pathology database, and a genome database. In a sixth example of the computing device, optionally including one or more or each of the first through fifth examples, the computing device is further configured to display on the screen a specific segment of the timeline of patient medical information in response to receiving a natural language input from a user, where the computing device is configured, in response to receiving the natural language input, to identify a patient condition-specific event in the natural language input, identify a temporal relationship between the patient condition-specific event and one or more other events in the timeline, and navigate to the specific segment based on the identifying. In a seventh example of the computing device, optionally including one or more or each of the first through sixth examples, the computing device is further configured to adjust the timeline by adding and/or removing one or more symbols of the plurality of symbols based on a specialization of a user viewing the timeline currently.

The disclosure also provides support for a method, comprising: receiving a natural language input from a user, identifying a patient condition-specific event in the natural language input, identifying a temporal relation between the patient condition-specific event and one or more other events in a patient information timeline of the patient, navigating to a specific segment of the patient information timeline based on the identifying of the temporal relation, and displaying the specific segment of the patient information timeline on a display device. In a first example of the method, the patient information timeline includes a respective representation of the one or more other events ordered by time, and further includes representations of additional events ordered by time. In a second example of the method, optionally including the first example, the patient condition-specific event identified in the natural language input is not one of the one or more other events or additional events included in the patient information timeline. In a third example of the method, optionally including one or both of the first and second examples, the method further comprises: generating the patient information timeline by ingesting patient data from a plurality of data sources, identifying and extracting relevant patient condition-specific medical events in the patient data, generating a representation of each relevant patient condition-specific medical event, and displaying each representation in a time-ordered fashion. In a fourth example of the method, optionally including one or more or each of the first through third examples, ingesting patient data from the plurality of data sources comprises ingesting patient data from one or more of a picture archiving and communication system, a radiology information system, an electronic medical record database, a pathology database, and a genome database. In a fifth example of the method, optionally including one or more or each of the first through fourth examples, identifying and extracting the relevant patient condition-specific medical events in the patient data comprises applying natural language processing to the patient data to generate processed patient data and performing medical ontology inferencing on the processed patient data.

The disclosure also provides support for a method, comprising: generating a patient information timeline including a plurality of elements each visually representing a patient condition-specific medical event, record, and/or report in a time-ordered fashion, adjusting the timeline by adding and/or removing one or more elements of the plurality of elements based on a specialization of a user viewing the timeline currently, and displaying the adjusted timeline on a display device. In a first example of the method, the plurality of elements of the timeline and of the adjusted timeline are organized into lanes based on a category of the patient condition-specific medical event, record, and/or report represented by each element. In a second example of the method, optionally including the first example, generating the timeline comprises ingesting patient data from a plurality of data sources, identifying and extracting relevant patient condition-specific medical events, records, and/or reports in the patient data, generating an element for each relevant patient condition-specific medical event, record, and/or report, and displaying each element in the time-ordered fashion. In a third example of the method, optionally including one or both of the first and second examples, identifying and extracting the relevant patient condition-specific medical events, records, and/or reports in the patient data comprises applying natural language processing to the patient data to generate processed patient data and performing medical ontology inferencing on the processed patient data utilizing medical knowledge graphs. In a fourth example of the method, optionally including one or more or each of the first through third examples, ingesting patient data from the plurality of data sources comprises ingesting patient data from one or more of a picture archiving and communication system, a radiology information system, an electronic medical record database, a pathology database, and a genome database. In a fifth example of the method, optionally including one or more or each of the first through fourth examples, displaying each element in the time-ordered fashion comprise applying position constraints to each element to resolve any ambiguous element boundaries.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A computing device comprising a display screen, the computing device being configured to display on the screen a timeline of patient medical information including a plurality of symbols representing the patient medical information, wherein a symbol of the plurality of symbols is selectable to launch a details panel and enable a report that references the displayed patient medical information to be seen within the timeline, and wherein the symbol is displayed while the details panel is in an un-launched state.

2. The computing device of claim 1, wherein the plurality of symbols is displayed in one or more rows, each row corresponding to a different category of patient medical information, and the symbols in each row are ordered by time.

3. The computing device of claim 2, wherein each symbol of the plurality of symbols represents a patient medical event, a patient medical report, or patient medical data identified from one or more patient data sources.

4. The computing device of claim 1, wherein the details panel includes a summary of information included in the report.

5. The computing device of claim 1, wherein the patient medical information represented by the plurality of symbols relates to a specific patient medical condition and is originally stored in a plurality of separate data sources.

6. The computing device of claim 5, wherein the plurality of separate data sources comprises two or more of a picture archiving and communication system, a radiology information system, an electronic medical record database, a pathology database, and a genome database.

7. The computing device of claim 1, wherein the computing device is further configured to display on the screen a specific segment of the timeline of patient medical information in response to receiving a natural language input from a user, where the computing device is configured, in response to receiving the natural language input, to identify a patient condition-specific event in the natural language input, identify a temporal relationship between the patient condition-specific event and one or more other events in the timeline, and navigate to the specific segment based on the identifying.

8. The computing device of claim 1, wherein the computing device is further configured to adjust the timeline by adding and/or removing one or more symbols of the plurality of symbols based on a specialization of a user viewing the timeline currently.

9. A method, comprising:
receiving a natural language input from a user;
identifying a patient condition-specific event of a patient in the natural language input;
identifying a temporal relation between the patient condition-specific event and one or more other events in a patient information timeline of the patient;
navigating to a specific segment of the patient information timeline based on the identifying of the temporal relation; and
displaying the specific segment of the patient information timeline on a display device.

10. The method of claim 9, wherein the patient information timeline includes a respective representation of the one or more other events ordered by time, and further includes representations of additional events ordered by time.

11. The method of claim 10, wherein the patient condition-specific event identified in the natural language input is not one of the one or more other events or additional events included in the patient information timeline.

12. The method of claim 10, further comprising generating the patient information timeline by ingesting patient data from a plurality of data sources, identifying and extracting relevant patient condition-specific medical events in the patient data, generating a representation of each relevant patient condition-specific medical event, and displaying each representation in a time-ordered fashion.

13. The method of claim 12, wherein ingesting patient data from the plurality of data sources comprises ingesting patient data from one or more of a picture archiving and communication system, a radiology information system, an electronic medical record database, a pathology database, and a genome database.

14. The method of claim 12, wherein identifying and extracting the relevant patient condition-specific medical events in the patient data comprises applying natural language processing to the patient data to generate processed patient data and performing medical ontology inferencing on the processed patient data.

15. A method, comprising:
generating a patient information timeline including a plurality of elements each visually representing a patient condition-specific medical event, record, and/or report in a time-ordered fashion;
adjusting the timeline by adding and/or removing one or more elements of the plurality of elements based on a specialization of a user viewing the timeline currently; and
displaying the adjusted timeline on a display device.

16. The method of claim 15, wherein the plurality of elements of the timeline and of the adjusted timeline are organized into lanes based on a category of the patient condition-specific medical event, record, and/or report represented by each element.

17. The method of claim 15, wherein generating the timeline comprises ingesting patient data from a plurality of data sources, identifying and extracting relevant patient condition-specific medical events, records, and/or reports in the patient data, generating an element for each relevant patient condition-specific medical event, record, and/or report, and displaying each element in the time-ordered fashion.

18. The method of claim 17, wherein identifying and extracting the relevant patient condition-specific medical events, records, and/or reports in the patient data comprises applying natural language processing to the patient data to generate processed patient data and performing medical ontology inferencing on the processed patient data utilizing medical knowledge graphs.

19. The method of claim 17, wherein ingesting patient data from the plurality of data sources comprises ingesting patient data from one or more of a picture archiving and communication system, a radiology information system, an electronic medical record database, a pathology database, and a genome database.

20. The method of claim 17, wherein displaying each element in the time-ordered fashion comprise applying position constraints to each element to resolve any ambiguous element boundaries.
